(19) United States
(12) Patent Application Publication
(10) Pub. No.: US 2005/0256540 A1
(43) Pub. Date: Nov. 17, 2005

(54) Apparatus and method for movement of electricity or fluid into and out of the body

(76) Inventors: Theodore A. Silver, New York, NY (US); David J. Wood, Port Washington, NY (US)

Correspondence Address: David Aker, 23 Southern Road, Hartsdale, NY, 10530, US

(21) Appl. No.: 11/068,508

(22) Filed: Feb. 28, 2005

Related U.S. Application Data
(60) Provisional application No. 60/548,045, filed on Feb. 26, 2004.

(51) Int. Cl. A61N 1/18

(52) U.S. Cl. 607/3

(57) ABSTRACT

An apparatus for communication between a site within a body deep to the skin and a source, depot, reservoir, device, machine, or power source external to the body via the mouth of the body, comprising an implant for placement in tooth-supporting bone, the implant having a first portion extending into the mouth external to the tooth-supporting bone, a second portion for being embedded in the tooth-supporting bone, and an opening extending along the implant from the first portion to the second portion; and a conduit for coupling to the second portion of the implant when the implant is implanted in the tooth-supporting bone so that the conduit is in communication with the opening, the conduit being for extending from the implant to the site in the body.

This application claims priority from provisional patent application Ser. No. 60/548,045 filed on Feb. 26, 2004, the entire contents of which are incorporated herein by reference.

### BACKGROUND OF THE INVENTION

1. Field of the Invention

The present invention relates to apparatus and methods, for the movement of electricity or fluid into and out of the human body. More particularly, it is specifically directed to mediating such movement without traditional cannulae or catheters that pierce the skin and create significant risk of infection.

2. Background Art

Movement of fluid into and/or out of the blood vessels or other spaces deep to the skin is a necessary incident to many therapies including, most commonly, the intravenous administration of medication and electrolytic fluid. Parenteral nutrition/hyperalimentation requires that fluid move into the vascular system from a reservoir outside the body. In the process of renal dialysis, fluid is removed from the body, processed, and returned.

Less common but very significant are therapies requiring the movement of electricity from a power source outside the body to some site within. Indeed, any mechanism of moving electricity into the body without creating a permanent/long term opening in the skin will facilitate therapies, treatments, and devices not now available.

Notably, Left Ventricular Assistance Devices (LVADs) are implantable electrical pumps that offer effective treatment of heart failure. Unlike pacemakers, they require such electrical power as cannot be provided long term by a battery located within the body. Rather, the battery must be located outside the body and subject to frequent replacement or recharge. (The battery might be located inside the body but must then be subject to frequent recharge by a source outside the body.)

Where fluid or electricity is to be moved from a source outside the body to blood vessels, machinery, reservoirs or other sites within the body, prior art resorts to needles, cannulae, and catheters that traverse the skin and so create an opening between the environment and the site to which flow is directed. If the process is to be conducted over a long term the opening remains in place and presents a risk of serious infection. Modern protocol requires that the ordinary intravenous catheters inserted for fluid and medication infusion (most commonly in a vein at or near the wrist) be changed every three days in order to avoid infection.

Hyperalimentation, chemotherapy, long-term intravenous infusion, and frequent, repeated blood drawing are often facilitated by indwelling catheters called, more specifically, “tunneled central venous catheters” and “peripherally inserted central catheters” (PICCs). Once placed, a tunneled central venous catheter presents access ports at the chest wall. It penetrates the skin of the thoracic wall and then tunnels upward under the skin to reach the superior vena cava. The PICC, once placed, runs through the skin (piercing the skin, sometimes at the upper limb, to enter the brachial vein) and then reaches the subclavian vein (and, perhaps, the superior vena cava). Both tunneled central venous catheters and PICCs have infection as a chief cause of morbidity and mortality.

LVADs derive their electrical supply from external “drive lines” that penetrate the abdominal wall and then run upward in the thorax, deep to the skin, to reach the device near the heart. Some forty percent of patients with LVADs die of infection, due chiefly to the presence of the drive lines. For that reason, LVADs are normally implanted only in patients with “end-stage” heart failure (usually awaiting or recovering from transplant). For those with serious heart failure that is not “end-stage”, the risk of infection is so great as to overcome the promised benefit. The Jarvic ventricular assist device relies on a “skull pedestal” that brings electricity into the body via the scalp (the vascularity of which, it is said, tends to resist infection).

All processes requiring the long term delivery of fluid or electric current to the inside of the body would be rendered safer and more useful by a device that would mediate the delivery without the need for ongoing communication, through the skin, between environment and bodily interior. Prior art offers no such device.

Furthermore, some conditions (diabetes and hemophilia, for example), require not a long term cannula or indwelling catheter but regular and repeated subcutaneous, intramuscular or intravenous injection by needle and syringe. Injection does not raise any extraordinary risk of infection, but it is painful and inconvenient (especially for children). Repeated venupuncture (as is necessary, for example, to renal dialysis and to chronic conditions that require repeated intravenous infusion or blood sampling) damages the peripheral veins; access to usable veins often becomes enormously problematic. Patients whose conditions require, regularly and repeatedly, that fluid be moved between the inside and outside of the body would benefit from a device that mediates the movement without the need for venupuncture or injection. Prior art offers no such device.

### SUMMARY OF THE INVENTION

It is an object and feature of this invention to create on an ongoing or regular/intermittent basis, an avenue for flow of electricity or fluid between some source or device outside the human body to some vessel, organ, space, position, system, reservoir or device located inside the body (meaning any site deep to the skin), such to be achieved without piercing the skin and so without creating, for periods long or short, any open communication between skin and outside environment.

It is a further object and feature of this invention to eliminate, substantially, the risk of infection associated with “cannulae,” “peripherally inserted central catheters,” “tunneled central venous catheters,” “indwelling catheters” and related devices now devoted to introducing electricity or fluid to the inside of the human body on long term bases.

It is a further object and feature of this invention to eliminate, substantially, the necessity for regular, repeated needle puncture associated with syringes, intravenous catheters, and related devices now used to facilitate regular, repeated injection of medication or other processes that require regular access to blood vessels or other spaces deep to the skin.

It is a further object and feature of this invention to provide a device that allows for the introduction of an ongoing supply of electricity needed and suited to the operation of prosthetic devices located inside the body/deep to the skin, without creating or maintaining an opening through the skin, and thus to substantially eliminate the serious and significant risk of infection now associated with such efforts.

It is a further object and feature of this invention to provide a device that allows for the movement of fluid between the outside of the body and a blood vessel(s) or other site(s) located anywhere deep to the skin, without opening the skin and thereby to (a) reduce or substantially eliminate the pain, inconvenience, vascular damage, inaccessibility and risk associated with repeated injection, piercing of skin, and puncture of blood vessel and, (b) in the case of permanent/long term delivery (or withdrawal) of fluid, substantially eliminate the serious and significant risk of infection now associated with such undertakings.

These objects and others are achieved in accordance with the invention. As is elaborated in the text and illustrations describing the preferred embodiments, the apparatus in accordance with the invention comprises, in a first tunnel drilled vertically downward through the mandible into which is driven, after the fashion of a screw, a hollow structure, roughly cylindrical in shape, composed of a non-reactive material, threaded on its outer surface similar to that of a dental implant footing/anchor. In a second tunnel of smaller caliber (drilled medially from the mandible's lateral aspect, laterally from the mandible's medial aspect, or upward from its inferior aspect so positioned and directed as to cause the internal ends of the two tunnels to communicate), with the device implanted (as discussed below) the second narrower tunnel (a) creates communication and continuity through the mandible of conductors or piping, while (b) preventing damage to or extrusion of the device by the downward force potentially imposed on the device when the patient bites or chews.

If the invention is to be used for the introduction of electricity there may be fitted into the vertically oriented sleeve a coaxial conductor (hereinafter “coaxial insert” or “insert”), sealed to the sleeve against microbes via dental sealant/cement, flared at its upper end after the fashion of a standard dental implant abutment to create an anti-microbial seal between surrounding gingival tissue and the coaxial unit, presenting at its upper end a terminus to the mouth and at its lower end a terminus to the bottom of the first tunnel. To the lower terminus, where the two tunnels coincide, another terminus may be connected from which the conducting system proceeds through the smaller tunnel to the lateral, medial, or inferior aspect of the mandible (depending on the orientation of the second tunnel). From that point the conducting system is directed to the site at which power is to be supplied, coursing, preferably, always, deep to the skin. At the mouth, the male terminus may be connected to an upper finale terminus from which the conducting system proceeds out of the mouth to the power source (via some convenient route involving a skin tunnel placed beneath the lower lip or at the posterior aspect of the cheek (near the ear).

If the apparatus in accordance with the invention is to be used for fluid flow, the vertically oriented sleeve may be fitted with valved tubing, sealed to the sleeve against microbes by dental sealant/cement, and connected outside the body to the relevant source, reservoir, or device. The tubing passes through the larger tunnel, on into the smaller one and thence from the lateral or inferior aspect of the mandible to the blood vessel or other site from which or to which flow is directed coursing, always, beneath the skin.

### BRIEF DESCRIPTION OF THE DRAWINGS

The foregoing aspects and other features of the present invention are explained in the following description, taken in connection with the accompanying drawings, wherein:

FIG. 1 shows a right lateral view of the mandible in sagital section, with a tooth removed and tunnel drilled in the resulting space, in accordance with the invention.

FIG. 2 shows a perspective view of a hollow cylindrical implant in accordance with the invention.

FIG. 3 shows a right lateral view of the mandible in sagital section, with the implant shown in FIG. 2 driven into the tunnel shown in FIG. 1.

FIG. 4 shows a sectioned view of a coaxial conductor driven into the implant shown in FIG. 2, which implant, in turn, is driven into the tunnel shown in FIG. 1.

FIG. 5 shows a top view of the coaxial conductor shown in FIG. 4.

FIG. 6 shows a right lateral view of the mandible in sagital section, with implant and coaxial conductor in place, and with a second tunnel drilled medially from the lateral aspect of the mandible, into which may be inserted an electrical conduit leading to some site within the body.

FIG. 7 shows a right lateral view of the mandible in sagital section with communication between the tunnel shown in FIG. 1 and the second tunnel shown in FIG. 6.

FIG. 8 shows a frontal view of gingival tissue surrounding manidbular tissue, with implant and coaxial conductor in place, and with the second tunnel of FIG. 6 carrying a conduit from the coaxial conductor to some site within the body.

FIG. 9 shows a left lateral view of the inside of the mouth with conductors entering/exiting it via either the oral orifice, a skin tunnel through skin under the lower lip, or a skin tunnel through the cheek, and a “dental electroface” removably attached (in “snap-on-snap-off” fashion) to an artificial tooth.

FIG. 10A shows a sectioned view of a first (vertical) fluid conduit/pipe/tube threaded on its external surface, driven into the implant shown in FIG. 2 which implant, in turn, is driven into the tunnel shown in FIG. 1.

FIG. 10B shows, in perspective, a coupler that may connect the first conduit to a second conduit in accordance with the invention (as shown in FIG. 11).

FIG. 11 shows a frontal view of gingival tissue surrounding manidbular tissue, with implant and first (vertical) fluid conduit/pipe/tube in place, and with the second tunnel shown in FIG. 6 carrying a second (horizontal) fluid conduit/pipe/tube connected at roughly a right angle to the first (vertical) conduit (via the coupling shown in FIG. 10B) that second conduit then to carry fluid (via additional conduits as necessary) to or from some site within the body.

FIG. 12 shows, in schematic fashion, electric current moving from a source outside the body through an electrical conduit that runs into the mouth, connected then to the coaxial insert (not shown in this schematic figure), thence to a second conduit (not shown in this schematic figure) and, ultimately, to an LVAD, all in accordance with the invention.

### DETAILED DESCRIPTION OF THE PREFERRED EMBODIMENTS

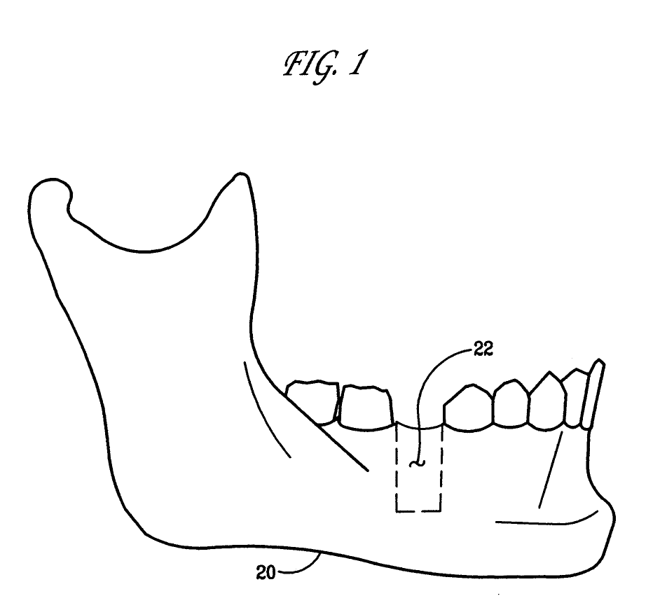

Referring to FIG. 1, a premolar/bicuspid or molar (or other) tooth has been removed from the mandible 20 (on right or left side). In the resulting space there is drilled downward through the mandible a vertically oriented tunnel 22, 10 mm to 15 mm deep and 4 mm to 7.5 mm in diameter (or of such other depth and diameter as suits the purpose described in the next sentence).

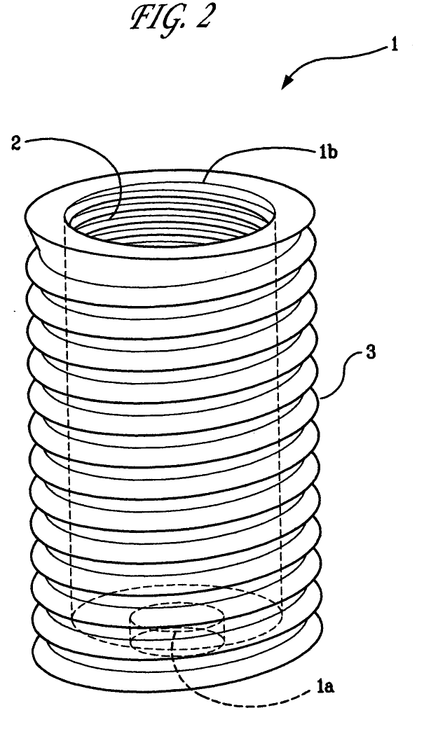

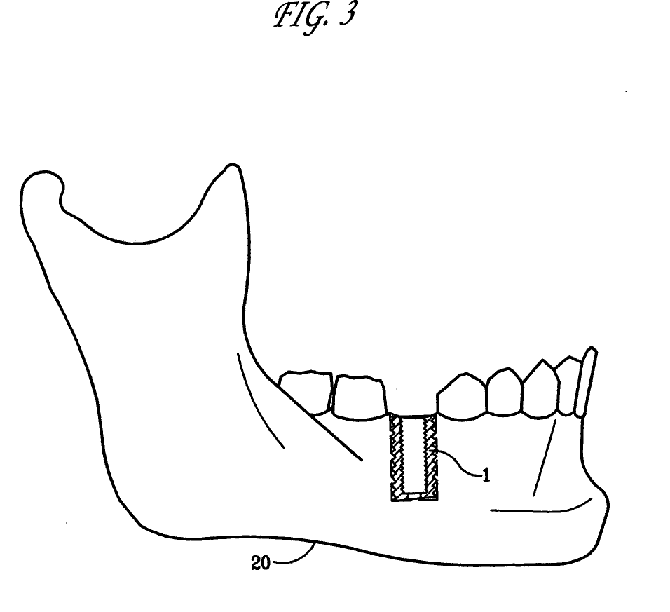

Referring to FIG. 2, driven into that tunnel, after the fashion of a screw, is a hollow entity or screw 1, roughly cylindrical in shape, open at both ends (the lower end 1A having a smaller diameter than the upper end 1B), composed of titanium, platinum or other non-reactive material. Its outer surface is threaded, as shown at 3 to facilitate fixture within the vertical tunnel 22 as shown in FIG. 3, with subsequent bone growth around it creating such a seal between sleeve and bone as does not permit the passage of microbes from the mouth or bodily exterior.

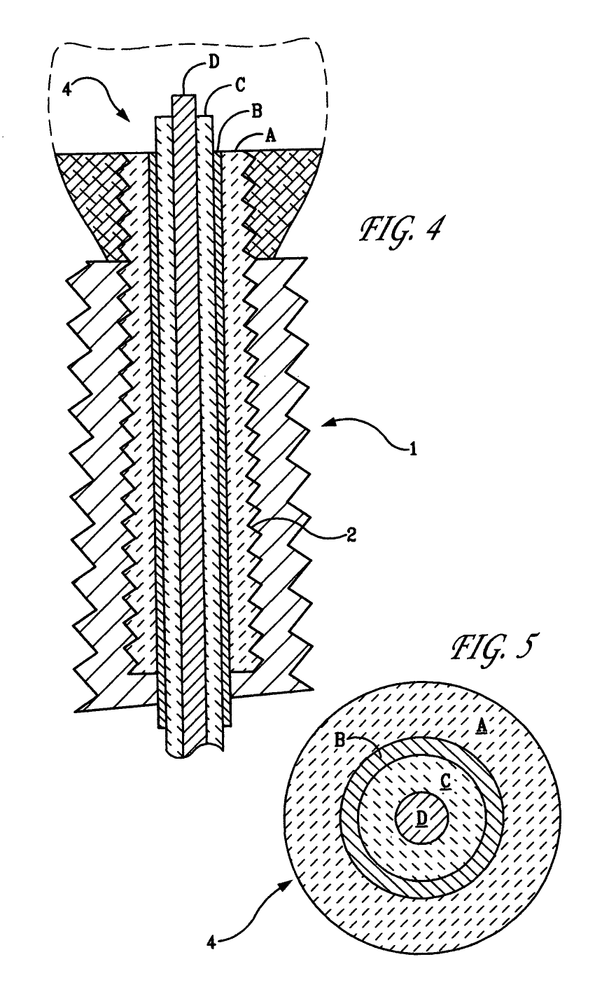

Referring to FIG. 4, the inner hollow aspect of the inserted component or screw 1 is also threaded, as shown at 2 and functions as a sleeve into which is driven, also after the fashion of a screw, a threaded cylindrical coaxial conductor or coaxial insert 4, which together with dental sealant/cement, creates such a seal between coaxial insert and sleeve, as does not permit the passage of microbes from the mouth or bodily exterior.

Referring to FIG. 4 and FIG. 5, the coaxial insert 4 is layered in composition from its external surface inward with a layer A of an insulating or dielectric material (plastic or ceramic for example) threaded, as just described, a conductor layer B, a layer of an insulating or dielectric material C, and a layer or core of a second conductor D.

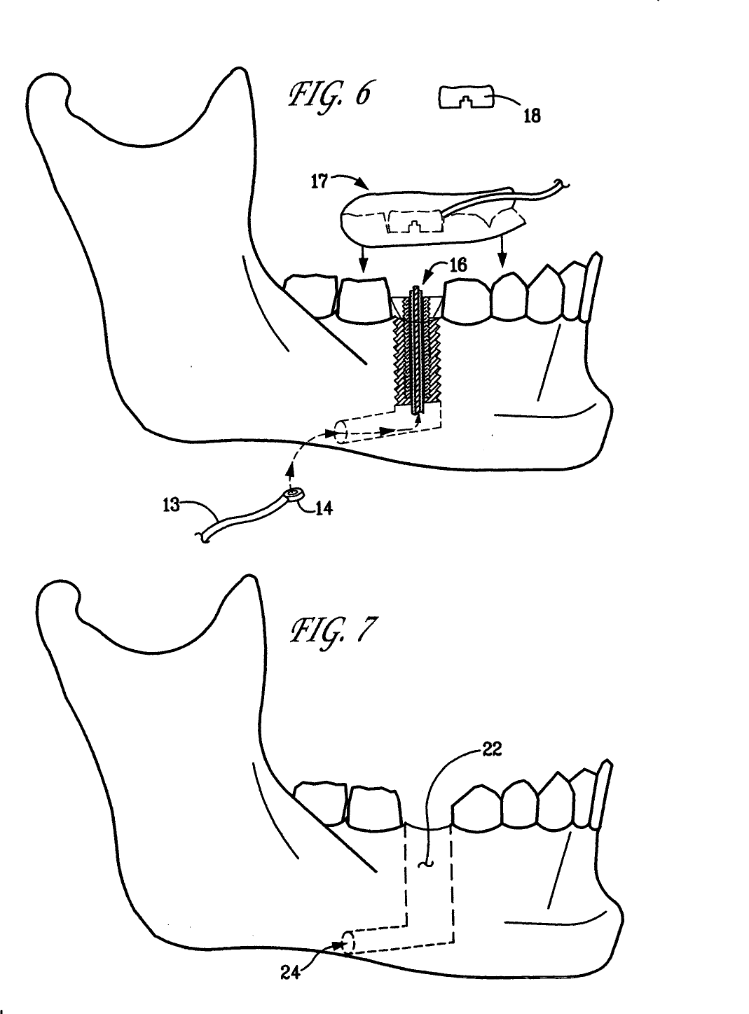

The coaxial insert 4 protrudes through the sleeve at both its upper end and its lower ends (FIG. 4), thus presenting (a) an upper male coaxial terminus to the mouth and a lower male coaxial terminus to the mandibular interstitium (FIG. 6).

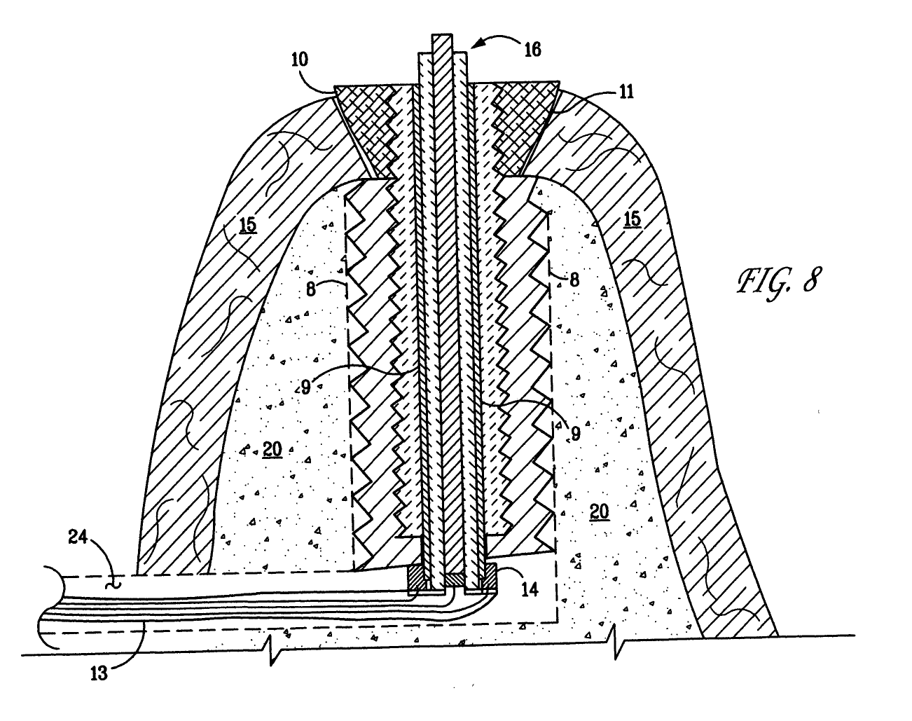

Referring to FIG. 7, a horizontally oriented tunnel 24 is drilled medially through the mandible), with the antero-posterior and infero-superior positions of horizontal and vertical tunnels corresponding sufficiently to create communication between the medial end of the horizontal tunnel 24 and the inferior end of the vertical tunnel 22. (Alternatively the tunnel could be drilled laterally from the medial aspect of the mandible to join the vertical tunnel.)

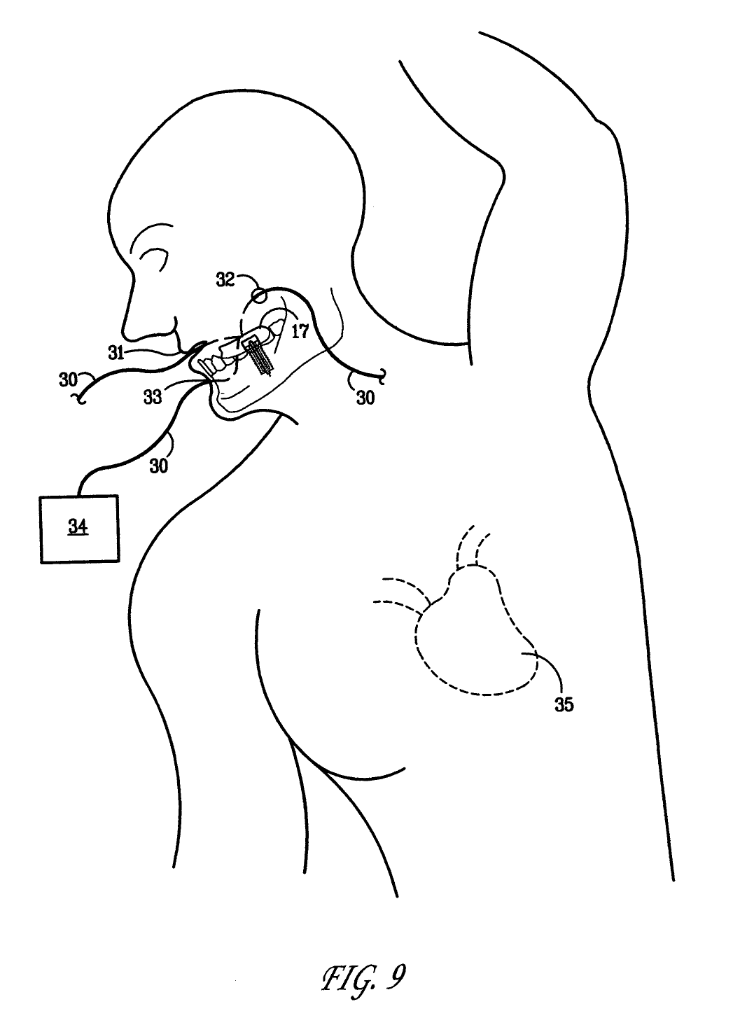

Referring to FIG. 6, in the horizontal tunnel 24 there is placed a second coaxial conductor 13, that presents to the lower coaxial male terminus a female coaxial terminus 14, threaded on its internal surface (roughly after the fashion, for example, of a screw-on bottle top or light bulb socket). Free space between horizontal conductor and horizontal tunnel (FIG. 8) is filled, as necessary, by application of a substance(s) that replaces bone or stimulates its formation, such as the “putty” or “bone in a bottle” or “bone formation powder” familiar to dentistry.

Referring to FIG. 8, the male terminus at the bottom of insert 4, is fitted into the female terminus 14 by screwing, in watertight fashion. Corresponding to the male terminus, the female terminus is layered in composition from its outermost to innermost aspects with: an insulating or a dielectric material (plastic or ceramic for example) threaded, as just described, to facilitate connection between male and female termini; conductor first conductor; layer 3 an insulating or dielectric material, and layer 4 a second conductor.

The device is installed in two parts through two separate tunnels, a wider one and a narrower one, as just described in order to (a) create communication and continuity through the mandible of the conductive pathway while (b) preventing damage to or extrusion of the device by the downward force potentially imposed on it when the patient bites or chews.

In the mouth, the protruding male terminus of insert 4 may be flared, after or with the fashion of a standard dental implant abutment 10 so to create an antimicrobial seal 11 between coaxial insert and gingival tissue 15, wherefore, all arrangements just described create a single coaxial conductive system extending from the male terminus 16 in the mouth through the upper aspect of the mandible, emerging at the lateral aspect of the mandible deep to the skin, with antimicrobial seals between (a) mandible and sleeve at 8, (b) sleeve and coaxial insert 9, and (c) coaxial conductor and gingival tissue at 11.

As illustrated in FIG. 6 and in FIG. 9, in the mouth a dental electro-interface 17, which may be a removable (“snap-on-snap-off”) component composed of acrylic (blow molded, or thermal molded, for example), form fitted after the fashion of a prosthetic dental lower partial plate/ridge/retainer, serves as female terminus, and sits securely over (a) several natural teeth (or such prosthetic substitutes as may characterize the patient's dental status), and (b) the upper male coaxial terminus 16, where it presents to the upper male terminus an upper female coaxial terminus. A prosthetic tooth 18 is placed over male terminus 16 when electro-interface 17 is not in use.

From the upper female terminus of electro-interface 17 just described, a coaxial conductor 30 (or other arrangement that embodies (at least) two insulated electrical leads) proceeds out of the mouth via some suitable route which might, for example, run (a) directly through the oral orifice or mouth as shown at 31, or (b) through a skin tunnel (like the “piercings” that accommodate jewelry) traversing the mucosa, muscles, subcutaneous tissues and skin situate between (i) the maxillary and mandibular bones (the buccal/“cheek” area) as shown at 32, or (ii) the lower labia and mental protuberance (between the “lower lip” and “chin”), as shown at 33, all as illustrated in FIG. 9.

The coaxial insert joins a cable (or other arrangement that embodies at least two insulated electric leads), which runs from its exit site at the mouth to a power source (such as a battery) 34, worn outside the body.

At the lateral end of the horizontal tunnel 24, the horizontally oriented coaxial conductor 14 presents a male or female end suitable for connection to a reciprocal connector (or it may present any conductive system that embodies at least two insulated electrical leads/conductors) which, in turn, will extend the conducting system to another internal area of the body, such as for example 35, by coursing, preferably always, deep to the skin.

The embodiment thus facilitates the introduction of electric power from (a) a battery source worn outside the body to an electrical device inside the body, or (b) another current source located outside the body (derived, for example, from a power supply connected to household current) into the body for the purpose of charging/recharging a battery located within the body which, in turn, directly powers an electrical device inside the body, all by a route which, unlike all other methods and devices now directed to that same purpose, leaves no open communication between the outside environment and any space deep to the skin, so that the usual risk of lethal infection is substantially eliminated.

Fluid Flow

In the preferred embodiment, in the manner described with respect to FIG. 1, FIG. 3, and FIG. 6 above, a premolar/bicuspid or molar (or other) tooth is removed from the mandible (on right or left sides). In the resulting space there is drilled downward through the mandible a vertically oriented tunnel, 10 to 15 mm deep and 4 to 7.5 mm in diameter (or of such other depth and diameter as suits the purpose described in the next sentence). Driven into that tunnel, after the fashion of a screw, is a hollow entity, roughly cylindrical in shape, open at both ends (the lower end having a smaller diameter than the upper end), composed of titanium, platinum or other non-reactive material. Its outer surface is threaded to facilitate fixture within the vertical tunnel as just described, with subsequent bone growth around it creating such a seal between sleeve and bone as does not permit the passage of microbes from the mouth or bodily exterior.

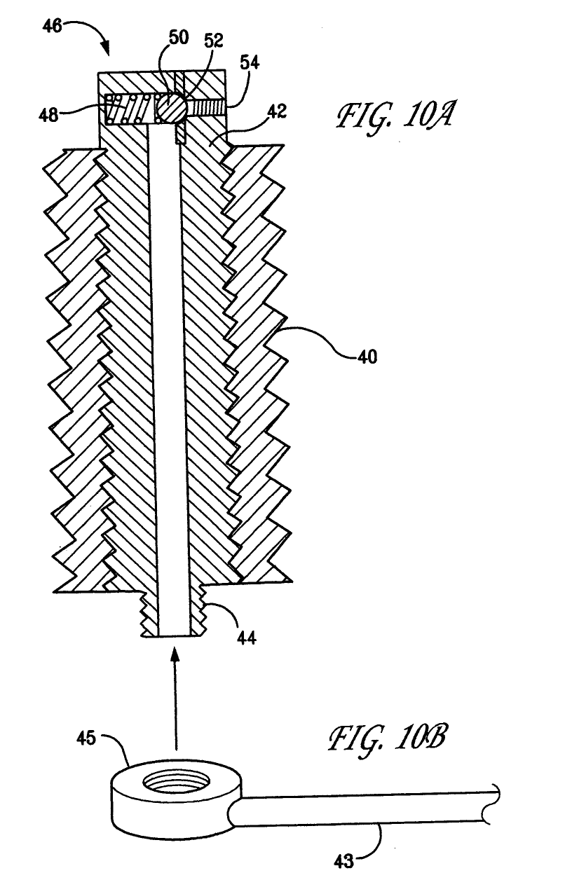

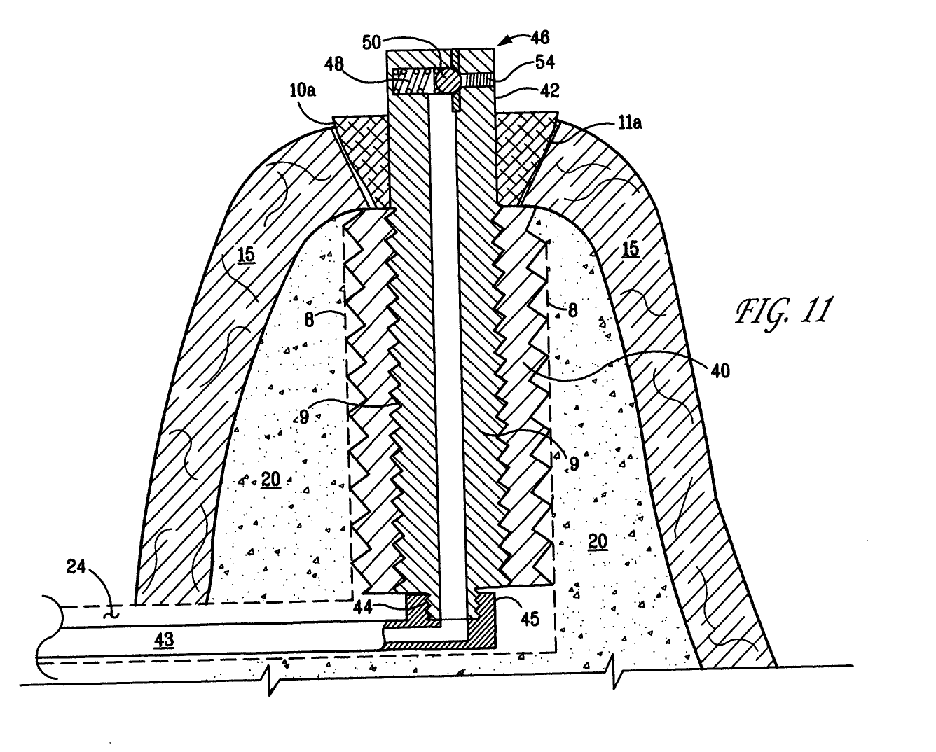

Referring to FIG. 10A, the inner hollow aspect of the inserted component is also threaded and functions as a sleeve 40 into which is driven, also after the fashion of a screw, a cylindrical gold fluid conduit/pipe/tube (pipe 42 hereinafter), threaded on its outer surface, which, together with dental sealant/cement creates such a seal between pipe and sleeve as does not permit the passage of microbes from the mouth or bodily exterior.

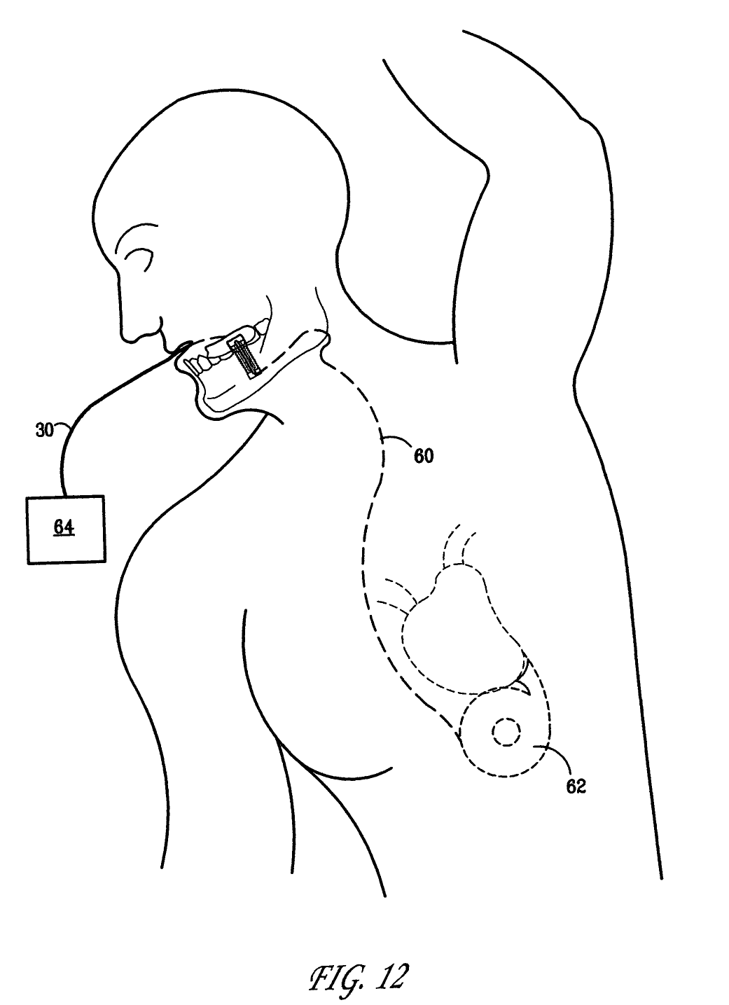

Referring also to FIG. 11 (which has many of the same reference numerals as FIG. 6, indicating like components), at its upper end the pipe 42 protrudes above the level of the mandible and sleeve 40 and is flared, after the fashion of a standard dental implant abutment 10A so to create an antimicrobial seal 11A between pipe and gingival tissue. At its lower end the gold pipe 42 narrows in caliber so to create a downward projecting threaded nipple 44 protruding through the sleeve 40.

A horizontally oriented tunnel 24 is drilled medially through the mandible, with the antero-posterior and infero-superior positions of horizontal and vertical tunnels corresponding sufficiently to create communication between the medial end of the horizontal tunnel and the inferior end of the vertical tunnel 24. (Alternatively, the tunnel might be drilled laterally from the medial aspect of the right side of the mandible to join the vertical tunnel.) In the horizontal tunnel 24 there is placed a second pipe 46 made of platinum, gold, or other hard non-reactive substance forming, roughly, a right angle with pipe 42 9 at a female coupling 45) at the medial end of the horizontal tunnel 24, where the horizontal tunnel 24 communicates with the vertical tunnel 22.

At that same point, the upward pointing end of the “elbow” receives the lower end of the vertical pipe by screwing, so that a continuous watertight system of piping/tubing runs from the upper surface of the mandible into the mandibular interstitium and then, laterally to the mandibles lateral aspect. Free space between the horizontal pipe and horizontal tunnel is filled, as necessary, by application of a substance(s) that replaces bone or stimulates its formation, such as the “putty” or “bone in a bottle” or “bone formation powder” familiar to dentistry.

The apparatus is installed in two parts through two separate tunnels, a wider one and a narrower one in order to (a) create communication and continuity through the mandible of piping while (b) preventing damage to or extrusion of the device by the downward force potentially imposed on it when the patient bites or chews.)

In the mouth, there is screwed to the upper end of the gold tube or pipe 42 a one-way valve system 46 permitting flow in the inward direction only. Valve system 46 may have a one-way check valve having a spring 48, a ball 50 and a concave wall 52 against which ball 50 rests to form a seal. An opening 54 may have internal threads to receive a threaded needle catheter (not shown). (Alternatively the tube is not open at its upper end, but rather carries a one-way valve as its upper end. Alternatively, the entire tubing system might be bi-luminal (“double barreled) each lumen equipped at its upper end with a valve, one permitting inward flow only and the other permitting outward flow only. Alternatively, for an application such as renal dialysis, in which fluid must move into and out of the vascular system, the patient might be fitted with two separate piping systems, one entering the mandible at each side of the mouth, one to carry fluid inward to the vascular system and one to carry it outward therefrom.

When flow is not to be initiated, the upper end of the pipe is covered by a form fitted (“snap-on-snap-off”) prosthetic tooth. When flow is to be initiated, the prosthetic tooth is removed. Suitable piping or tubing is connected to the valve and the valve is opened. The tubing will exit the mouth (in a manner analogous to that which is shown in FIG. 9, (a) directly through the oral orifice, or (b) through a skin tunnel (like the “piercings” that accommodate jewelry) traversing the mucosa, muscles, subcutaneous tissues and skin situate between (i) the maxillary and mandibular bones (the buccal/“cheek” area) or (ii) the lower labia and mental protuberance (between the “lower lip” and “chin”).

At the lateral end of the horizontal tunnel, the horizontally oriented tube is connected to additional piping/tubing which, in turn, will extend the conducting system to the vessel, space, site or device to which or from which fluid is to move coursing, preferably always, deep to the skin.

The embodiment thus facilitates the movement of fluid into and/or out of the body from or to some source, depot, reservoir or device outside the human body to some site, space, vessel, or device located inside the body all by a route which, unlike all other methods and devices now directed to that same purpose, leaves no open communication between the outside environment and any space deep to the skin, so that the pain, inconvenience, inaccessibility, and risk of lethal infection are substantially eliminated.

Preferred Applications

Electrical Conduction

A. As described above with respect to the prior art, Left ventricular devices (LVADs), (bearing the brand names, for example “Heartmate,” “Arrow LionHeart,” and “Debakey,”) require external “drive lines” to deliver power from an external battery pack. (Thoratec, the manufacturer of “Heartmate” has also manufactured a device driven pneumatically so that the abdomen is pierced not by electrical conductors but rather by hoses delivering pneumatic power which, in turn, is generated by electric machinery located outside the body). The electrical drive lines create a grave risk of infection (some 40% of patients dying of infection), this being the LVAD's chief drawback and disadvantage.

The Jarvic 2000 LVAD seeks to address the problem by directing current not through an abdominal piercing but through the scalp (whose vascularity is said to decrease the risk of infection). Undergoing testing in Europe (but not in the U.S.), the Arrow LionHeart LVAD seeks to solve the drive-line infection problem by operating on transcutaneous energy transfer/induction (TET). Shortcomings are likely to include (a) malfunction through electromagnetic interference, and (b) limitation as to the magnitude of power that can be introduced.

Referring to FIG. 112, in accordance with the present invention, in one preferred application of the preferred embodiment, conductors 60 emanating from the lateral aspect of the mandible (or from the medial aspect if the horizontal tunnel is drilled laterally from the medial aspect of the mandible toward the vertical tunnel) are tunneled under the skin and connected to the LVAD 62, in place of the drive-lines offered by prior art. Conductors emanating from the mouth (as described in the preferred embodiment) lead to a battery 64 (worn on the belt or in a pocket, for example), and the battery 64 powers the LVAD. (The LVAD is also equipped with an internal battery). Circuitry allows for current to run directly from the external battery to the LVAD and also for recharging of the internal battery from some other source (derived, for example, from household current). If, for some reason, the patient wished temporarily to be free of the external wiring, she would remove the dental electro-face, replace it with an artificial tooth, disconnect the wiring from the dental electro-interface, and remove both wiring and dental electro-interface from her mouth. The LVAD would then run on its internal battery, which would be subject to recharging when the connections were restored.

B. For any person who has lost the rectum (usually with some length of colon as well) and is thus consigned permanently to a colostomy bag, a second preferred application of the preferred embodiment powers a one-way peristaltic pump fixed to a synthetic colon that is drawn distally to an artificial (or residual/natural) anus located at or near the perineum. The synthetic colon, in turn, is bound by an airtight/watertight surgical connection proximally to the distal end of the disrupted colon, and distally to the artificial anus (or residual rectum if there be any that is for some reason non-attachable to the colon). The synthetic colon and peristaltic pump are equipped with a one-way valve system, with pump and valve system being electrically operated from outside the body, thus allowing the patient to control defecation and to eliminate quasi-normally through an artificial (or residual/natural) anal opening. A gauge/alarm/signal system (also electrically operated) alerts the patient to the status of filling at the synthetic colon and thus apprises him as to an appropriate time for elimination. (The synthetic colon will be readily accessible from the outside for cleaning by a “bidet” or similar device.)

In accordance with the preferred embodiment, conductors 60 emanating from the lateral aspect of the mandible (or from the medial aspect if the horizontal tunnel is drilled laterally from the medial aspect of the mandible toward the vertical tunnel) are tunneled under the skin toward the abdomen, through the abdominal subcutaneous tissues, through the abdominal muscles, into the abdominal cavity, and thus connected to the peristaltic pump, valve system, and gauge/alarm/signal system. Conductors 30 emanating from the mouth (as described in the preferred embodiment) lead to a battery (worn on the belt or in a pocket, for example), which powers the peristaltic pump, valve system, and gauge/alarm/signal system. The pump might also be equipped with an internal battery.

Circuitry allows current to run directly from external battery to the pump, valve system, gauge/alarm/signal system and, also, for recharging of the internal battery from some other source (derived, for example, from household current). If, for some reason, the patient wished temporarily to be free of the external wiring, she would remove the dental electro-interface, replaces it with an artificial tooth, disconnect the wiring from the dental electro-interface and remove both wiring and dental electro-interface from her mouth. The pump would then run on its internal battery, which would be subject to recharging when the connections were restored.

Circuitry leading from the peristaltic pump to the outside of the body can include a switch or switches (carried on the belt or in a pocket, for example). With the circuit closed (switch(es) “turned on”) the valve opens and the pump operates thus facilitating quasi-normal defecation through natural or artificial anal opening.

Surgical connection between the synthetic colon and the living tissue of true colon and might be accomplished by turning an external cuff in the colon and an internal cuff in the synthetic colon (or vice-versa) which at its proximal end would be composed of gold mesh. The internal cuff would then lock into the external cuff. Suturing the two aspects of the colonic cuff together with the gold mesh in between would then cause healing and sealing between the two aspects of the cuff, creating a continuous watertight/airtight channel of colon and synthetic colon. An analogous procedure would be performed at the rectum/anus, thus to create a continuous airtight/watertight canal from colon to anus, via synthetic colon equipped with a peristaltic pump.)

Fluid Flow

A. Persons whose kidneys malfunction or fail to function at all are treated regularly and repeatedly (several times per week) with the processes of hemodialysis (or, in some cases, peritoneal dialysis). Hemodialysis requires that fluid move outward from the patient's vascular system, then to the relevant machinery, and back to the vascular system. In order to facilitate the inward and outward flow the patient will be fitted with either (a) a bi-luminal (“double barreled”) piping system, each lumen equipped at its upper end with a valve, one permitting inward flow only and the other permitting outward flow only, or (b) two separate piping systems (one entering the mandible at each side of the mouth, for example), one to carry fluid inward to the vascular system and one to carry it outward.

B. Persons whose condition prevents them from feeding via the gastrointestinal tract are treated with parenteral nutrition/hyperalimentation which requires permanent or long term access to a large vein (such as the subclavian vein). In such a patient the pipe emanating from the lateral end of the mandible (as described in preferred embodiment B) is led by additional watertight/airtight coupling to a large vein such as the subclavian. Nutritional fluid/substance will flow from its reservoir outside the body through the piping system into the vein. Any outward flow of fluid that the process might necessitate will be facilitated either by (a) a biluminal (“double barreled”) piping system, each lumen equipped at its upper end with a valve, one permitting inward flow only and the other permitting outward flow only, or, as described above, (b) two separate piping systems (one entering the mandible at each side of the mouth, for example), one to carry fluid inward to the vascular system and one to carry it outward.

It should be understood that the foregoing description is only illustrative of the invention. Various alternatives and modifications can be devised by those skilled in the art without departing from the invention. Accordingly, the present invention is intended to embrace all such alternatives, modifications and variances that fall within the scope of the appended claims.

1. An apparatus for communication between a site within a body deep to the skin and a source, depot, reservoir, device, machine, or power source external to the body via the mouth of said body, said apparatus comprising:
an implant for placement in tooth-supporting bone, said implant having a first portion extending into the mouth external to said tooth-supporting bone, a second portion for being embedded in said tooth-supporting bone, and an opening extending along said implant from said first portion to said second portion; and
a conduit for coupling to said second portion of said implant when said implant is implanted in said tooth-supporting bone so that said conduit is in communication with said opening, said conduit being for extending from said implant to said site in said body.

2. The apparatus of claim 1, further comprising a one-way valve associated with said implant so as to allow fluid to flow in only one direction through said opening and along said conduit.

3. The apparatus of claim 1, further comprising an insert within said opening, said insert comprising at least one electrical conductor.

4. The apparatus of claim 3, further comprising an insulator for insulating said electrical conductor from said implant.

5. The apparatus of claim 1, in combination with a medical device implanted in the body at said site within the body.

6. The combination of claim 5, wherein said medical device is selected from a group consisting of a left ventricular assist device, a peristaltic pump, and an artificial heart.

7. The combination of claim 5, wherein said medical device comprises: a section of artificial colon; and a peristaltic pump associated with said section of artificial colon for propelling fecal/eliminable material along said section.

8. The apparatus of claim 1, further comprising: a member for being received within the mouth so as to couple to said first portion of said implant, said member having a passageway for coupling to said opening.

9. The apparatus of claim 8, further comprising means for releaseably coupling said member to said first portion of said implant.

10. The apparatus of claim 8, further comprising a conduit for connecting said device to said member.

11. The apparatus of claim 10, in combination with said device.

12. The apparatus of claim 11, wherein said device is selected from the group consisting of a source of electrical energy, a blood filtration (dialysis) device, an infusion device, or a blood sampling device.

13. A device that conducts electricity between the outside and inside of the human body via conductors that run from outside the body into the mouth, then to a coaxial insert that runs through a tunnel drilled in the mandible, then out of the mandible to a position beneath the skin and then under the skin to the site, device, or machinery to which electrical power is to be brought, with (a) antimicrobial seals between mandible and coaxial insert, and (b) airtight/watertight electrical connections (i) in the mouth, where the coaxial insert's superior end comes through the mandible, and (ii) at all places within the body, including the mandibular interstitium, where the conductive system is to be extended by additional connection.

14. The device of claim 13 with the coaxial insert driven into a hollow sleeve, threaded on its internal and external aspects which itself is driven into the mandible, with antimicrobial seals between (a) mandible and sleeve, and (b) sleeve and coaxial insert.

15. The device of claim 14 with the antimicrobial seals there referenced being facilitated (a) between sleeve and mandible by bone growth, and (b) between sleeve and coaxial insert by dental sealant/cement, and (c) at the mandible's upper surface, in the mouth, by a coaxial insert that is flared at its upper end to create/work as/work together with a dental implant abutment and gingival tissue in the mouth.

16. The device of claim 15 further comprising:
(a) a first tunnel drilled vertically into the mandible in which is carried a first coaxial insert, and
(b) a second narrower tunnel drilled medially from the mandible's lateral aspect or upward from the mandible's inferior aspect, or laterally from the mandible's medial aspect on one side, the second tunnel, carrying a second coaxial conductor/cable with the second tunnel in either case communicating with the first tunnel so to facilitate an airtight/watertight connection between the first coaxial insert and second coaxial conductor, thus to bring current from the outside of the body into the mouth, through the first coaxial insert situate in the first tunnel, through the second coaxial conductor/cable situate in the second tunnel and then, via tunneling beneath the skin, to the site to which electricity is to be brought.

17. The device of claim 16, further comprising:
(a) a hollow quasi-cylindrical sleeve threaded on its internal and external aspects driven after the fashion of a screw into the first tunnel, with bone growth creating an antimicrobial seal between sleeve and mandible, and
(b) a first coaxial insert threaded on its external aspect, that
(i) is driven, after the fashion of a screw, into the said sleeve,
(ii) at its upper end protrudes through the surrounding gingival tissue, above the level of the sleeve, and is flared, after the fashion of a standard dental implant abutment, thus creating an antimicrobial seal between coaxial insert and surrounding gingival tissue, and
(iii) at its lower end protrudes below the lower level of the sleeve, so to join the second coaxial conductor/cable by airtight/watertight connection where the second tunnel communicates with the first.

18. The device of claim 17, in which, further, the free space between horizontal conductor/cable and horizontal tunnel is filled by a substance(s) that replaces bone or stimulates its formation, such as the “putty” or “bone in a bottle” or “bone formation powder” familiar to dentistry.

19. The device of claim 18 including further, in the mouth a “dental electroface” comprising a removable component composed of acrylic (blow molded, or thermal molded, for example), form fitted after the fashion of a prosthetic dental lower partial plate/ridge/retainer, sits securely over
(a) several natural teeth (or such prosthetic substitutes as may characterize the patient's dental status), and
(b) the upper male coaxial terminus, where it presents to the upper male terminus an upper female coaxial terminus.

20. The device of claim 19, including further, a coaxial cable or set of conductors that lead from their airtight/watertight junction with the first coaxial conductor in the mouth, outward from the mouth to a power source, through a skin tunnel (like the “piercings” that accommodate jewelry) traversing the mucosa, muscles, subcutaneous tissues and skin situate between (i) the maxillary and mandibular bones (the buccal/“cheek” area) or (ii) the lower labia and mental protuberance (between the “lower lip” and “chin”).

21. The device of claim 13 configured for use in bringing electrical power to any connection with any ventricular assistance device.

22. The device of claim 13 configured for use in bringing electrical power to any artificial heart or any electrically powered device designed as a substitute for or adjunct to the heart.

23. The device of claim 13 configured for use in bringing electrical power to a one-way valved peristaltic pump that drives fecal material through a synthetic colon which, in turn, is joined by surgical airtight/watertight connection proximally to the severed end of the colon and distally to the rectum or anus.

24. A device that conducts fluid between some site, source, depot, reservoir, device, or machinery outside the body and some site, source, reservoir, depot, device or machinery inside of the human body via fluid conduit, tubing or piping that runs from the site, source, depot, reservoir, device, or machinery outside the body into the mouth, then to an insert implanted in a tunnel drilled through the mandible, which insert is or embodies a fluid conduit/pipe/tube (“pipe” hereinafter) that carries a one-way valve permitting inward flow only if fluid is to move inward to the body or outward flow only if fluid is to move outward from the body, then through the tunnel drilled in the mandible, then out of the mandible to a position beneath the skin and then under the skin to the site, depot, reservoir, device, or machinery from which or to which fluid is to be brought, with (a) antimicrobial seals between mandible and (b) airtight/watertight couplings (i) in the mouth, where the piping's superior end comes through the mandible, and (ii) at all places within the body, including the mandibular interstitium, where the piping is to be extended by additional connection.

25. The device of claim 24 with the pipe driven into a hollow sleeve, threaded on its internal and external aspects which itself is driven into the mandible, with antimicrobial seals between (a) mandible and sleeve, and (b) sleeve and coaxial insert.

26. The device of claim 25 with the antimicrobial seals there referenced being facilitated (a) between sleeve and mandible by bone growth, and (b) between sleeve and pipe by dental sealant/cement, and (c) at the mandible's upper surface, in the mouth, by a pipe that is flared at its upper end to create/work as/work together with a dental implant abutment and gingival tissue in the mouth.

27. The device of claim 26 including (a) a first tunnel drilled vertically into the mandible in which is carried a first pipe and (b) a second narrower tunnel drilled medially from the mandible's lateral aspect or upward from the mandible's inferior aspect, the second tunnel carrying a second pipe, with the second tunnel in either case communicating with the first tunnel so to facilitate an airtight/watertight coupling between the first pipe and the second pipe (by elbow connection if the two pipes run in different directions) thus to bring fluid from the outside of the body into the mouth, through a one way valve situate in the first pipe, through the second pipe which is positioned in the second tunnel and then, via tunneling beneath the skin, to the site to which (a) fluid is to be brought or (b) from which fluid is to be drawn.

28. The device of claim 27, further comprising:
(a) a hollow quasi-cylindrical sleeve threaded on its internal and external aspects driven after the fashion of a screw into the first tunnel, with dental cement or like substance creating an antimicrobial seal between sleeve and mandible, and
(b) a first pipe threaded on its external aspect, that is driven, after the fashion of a screw, into the said sleeve, and at its upper end
(i) protrudes through the surrounding gingival tissue, above the upper end of the sleeve, and
(ii) is flared, after the fashion of a standard dental implant abutment, thus creating an antimicrobial seal between piping and surrounding gingival tissue, and
(iii) at its lower end protrudes below the lower end of the sleeve, so to join the second pipe by airtight/watertight coupling where the second tunnel communicates with the first.

29. The device of claim 28 in which, further, the free space between horizontal piping and horizontal tunnel is filled by a substance(s) that replaces bone or stimulates its formation, such as the “putty” or “bone in a bottle” or “bone formation powder” familiar to dentistry.

30. The device of claim 29 including further, piping or tubing that leads from its airtight/watertight junction with the first pipe in the mouth outward from the mouth to a reservoir or depot through a skin tunnel (like the “piercings” that accommodate jewelry) traversing the mucosa, muscles, subcutaneous tissues and skin situate between (i) the maxillary and mandibular bones (the buccal/“cheek” area) or (ii) the lower labia and mental protuberance (between the “lower lip” and “chin”).

31. The device of claim 24 comprising first and second fluid conductors/pipes/tubes that bear two lumens each (“double-barreled”) one lumen equipped with a one way valve that permits inward flow only and one equipped with a one-way valve that permits outward flow only.

32. The device of claim 24 configured for infusion of electrolytic fluid and medication including insulin, “clotting factor (s)” used for treatment of hemophilia, and chemotherapeutic agents used to treat cancer.

33. The device of claim 24 configured for use in connection with at least one of parenteral nutrition or hyperalimentation.

34. The device of claim 24 configured for the withdrawal of blood.

35. The device of claim 24 configured for inward and outward flow between a vascular system and apparatus used for one of renal hemodialysis and peritoneal dialysis.

36. The device of claim 24 configured for delivery to/replenishment of/replacement of the hormone glucagon to a reservoir situated within a device, which device, in turn, (a) is implanted beneath the skin, (b) in contact with blood, (c) sensitive to blood glucose concentration, and (d) releases glucagon to the blood should such glucose levels fall too low, thereby avoiding “insulin shock,” and/or “hypoglycemia.”

37. A method for installing the apparatus of claim 1, comprising:
(A) forming a first tunnel vertically upward or downward through tooth-supporting bone;
(B) forming a second tunnel of smaller caliber through the same tooth supporting bone in such fashion as to cause the first and second tunnels to communicate at their internal ends, so to create a continuity of tunneling through the tooth-supporting bone, and
(C) placing within the first tunnel said implant and with the second tunnel said conduit so that said implant and said conduit are connected.

38. A method for installing the device of claim 13, comprising:
(A) forming a first tunnel vertically upward or downward through tooth-supporting bone;
(B) forming a second tunnel of smaller caliber through the same tooth supporting bone in such fashion as to cause the first and second tunnels to communicate at their internal ends, so to create a continuity of tunneling through the tooth-supporting bone, and
(C) placing within the first tunnel said implant and with the second tunnel said conduit so that said implant and said conduit are connected.

39. A method for installing the device of claim 24, comprising:
(A) forming a first tunnel vertically upward or downward through tooth-supporting bone;
(B) forming a second tunnel of smaller caliber through the same tooth supporting bone in such fashion as to cause the first and second tunnels to communicate at their internal ends, so to create a continuity of tunneling through the tooth-supporting bone, and
(C) placing within the first tunnel said implant and with the second tunnel said conduit so that said implant and said conduit are connected.
